SEQUENCE LISTING (7 sequences)

SEQ ID NO: 1 - DNA, 15 bp, Artificial Sequence
  Description of Artificial Sequence Primer
agcgggccggactca

SEQ ID NO: 2 - DNA, 22 bp, Artificial Sequence
  Description of Artificial Sequence Primer
taaccaccatcggatcaatgtc

SEQ ID NO: 3 - DNA, 20 bp, Artificial Sequence
  Description of Artificial Sequence Primer
cttgctctgttgcccaggct

SEQ ID NO: 4 - DNA, 21 bp, Artificial Sequence
  Description of Artificial Sequence Primer
ctcaccgtgctggaagacact

SEQ ID NO: 5 - DNA, 16 bp, Artificial Sequence
  Description of Artificial Sequence Probe
tctgcccacagtacct

SEQ ID NO: 6 - DNA, 15 bp, Artificial Sequence
  Description of Artificial Sequence Probe
ctgcccacggtacct

SEQ ID NO: 7 - DNA, 16 bp, Artificial Sequence
  Description of Artificial Sequence Probe
agacccaggcccacat

(19) United States
(12) Patent Application Publication
(10) Pub. No.: US 2005/0153300 A1
(43) Pub. Date: Jul. 14, 2005

(54) Methods and compositions for the detection of mucolipidosis IV mutations

(75) Inventors: Weimin Sun, Irvine, CA (US); Feras Hantash, Dana Point, CA (US)

Correspondence Address: FOLEY & LARDNER, P.O. BOX 80278, SAN DIEGO, CA, 92138-0278, US

(73) Assignee: Quest Diagnostics Incorporated

(21) Appl. No.: 10/754,446

(22) Filed: Jan. 9, 2004

(51) Int. Cl. C12Q 1/68; C07H 21/04

(52) U.S. Cl. 435/6; 536/24.3; 536/25.32

(57) ABSTRACT

The present invention provides a simple high-throughput assay for detecting Mucolipidosis IV mutant sequences (IVS3-2A>G, and/or the del6.4 kb) and optionally wildtype sequences in the same assay using probes with labeled with detectable labels, amplification with a DNA polymerase and acquisition of fluorescence in real time. The assay is automated, high throughput and cost effective, since two mutations and a wildtype sequence can be detected in a single reaction, as opposed to separate reactions.

### FIELD OF THE INVENTION

The present invention relates to compositions and methods for the detection of mucolipidosis IV mutations.

### BACKGROUND OF THE INVENTION

The following description of the background of the invention is provided simply as an aid in understanding the invention and is not admitted to describe or constitute prior art to the invention.

Mucolipidosis Type IV (ML IV) is a rare, neurodegenerative, autosomal recessive disorder. ML IV is a lysosomal storage disease that results in the excessive transport of membrane components (mucopolysaccharides and lipids) to lysosomes compared with normal cells. ML IV cells exhibit normal lysosomal hydrolases which eventually degrade the abnormally transported membrane components. There is no massive and progressive accumulation of membrane components as is the case in most other lysosomal storage disorder. Over 80% of patients with ML IV are of Ashkenazi-Jewish descent. The estimated carrier rate of ML IV is 1:100 with an incidence of about 1:40,000. Two mutations in the MCOLN1 gene account for 95% of the Ashkenazi Jewish ML IV alleles. An A>G transition (IVS 3-2 A>G), which causes a splice site mutation in the acceptor site of intron 3 is found in 72% of Ashkenazi ML IV alleles. A 6.4 kb deletion that includes exons 1 to 7 of the MCOLN1 gene (GenBank accession no. AF287270) is found in 23% of Ashkenazi ML IV alleles. See e.g., Bach (2001)Molecular Genetics and Metabolism 73: 197-203; Bargal et al., (2001) Human Mutation 17:397-402; Bargal et al., (2000) Nature Genetics 26: 118-121; Bassi et al., (2000) Am. J Genet. 67:1110-1120; and Sun et al., (2000) Human Molecular Genetics 9: 2471-2478.

The MCOLN1 gene is located on chromosome 19p 13 and encodes the mucolipin-1 protein. Mucolipin-1 is a membrane protein with 6 transmembrane domains, a channel pore region, and a serine lipase motif. Mucolipin-1 shares homology with the calcium channels of the transient receptor potential (TRP) cation gene family. ML IV represents the first example of a neurological disease caused by a TRP related channel.

The clinical picture is determined primarily by the extent of the storage, explaining the minimal deterioration and that most patients remain in a steady state. The majority of ML IV patients never develop the ability to speak or walk and remain at a developmental age of 1-2. ML IV is characterized by psychomotor retardation and opthalmological abnormalities including corneal opacities and retinal degeneration. There is considerable heterogeneity in the clinical symptoms and severity of the disease. Improved methods for diagnosing genetic disposition to ML IV are needed.

### SUMMARY OF THE INVENTION

The present invention provides methods and compositions for determining the presence of ML IV mutant sequences and/or wildtype sequences in a sample. In particular, substantially isolated oligonucleotides for detecting ML IV mutant or wildtype sequences in nucleic acids in a sample by amplification methods are described herein. The present invention provides a specific sensitive method that allows separate or simultaneous detection of multiple ML IV mutant sequences in the same assay. The methods and compositions for detecting the presence of mutant or wildtype ML IV sequences can be used for genetic counseling of individuals at risk for ML IV or at risk to having offspring afflicted with this disease.

In various embodiments of the present invention, oligonucleotide primers and probes are used in the methods described herein to provide the ML IV assay. Thus, in certain embodiments, the invention relates to primer sequences that can optionally be used together to amplify nucleic acid sequences for detection of two ML IV mutant sequences and a wildtype sequence which may be present in a sample. Primer based amplification and detection of amplified sequence by hybridization with fluorescently labeled probes are conducted together using a real time PCR format.

In additional embodiments, the invention relates in part to probe nucleic acids that can be conjugated to a detectable label, preferably, a fluorophore, and more preferably a donor fluorophore and a quencher moiety. The donor and quencher can located at or near the 5′ and 3′ end of the oligonucleotides. The quencher moiety may optionally be an acceptor fluorophore selected to emit fluorescence as a result of donor fluorophore emission.

In a first aspect, the invention relates to a compositions of oligonucleotides for amplification of the MCOLN1 gene regions. In one embodiment, the composition comprises one or more oligonucleotides comprising a sequence complementary to the coding or non-coding strand of the MCOLN1 gene wherein the sequence includes a sequence consisting essentially of: 5′-AGC GGG CCG GAC TCA-3′ (SEQ ID NO. 1), 5′-TAA CCA CCA TCG GAT CAA TGT C-3′ (SEQ ID NO. 2), 5′-CTT GCT CTG TTG CCC AGG CT-3′(SEQ ID NO. 3), or 5′-CTC ACC GTG CTG GAA GAC ACT -3′ (SEQ ID NO. 4), or a complementary sequence thereof. In another, the composition comprises one or more oligonucleotides wherein the sequence is SEQ ID NO. 1, SEQ ID NO. 2, SEQ ID NO. 3; or SEQ ID NO. 4, or a complementary sequence thereof.

In another aspect, the invention relates to a oligonucleotides as probes for detecting ML IV mutant or wildtype sequences amplified from the MCOLNl gene. In one embodiment, the oligonucleotide comprises a sequence complementary to the coding or non-coding strand of the MCOLN1 gene wherein the sequence includes a sequence consisting essentially of: 5′-TCTG CCC ACA GTA CCT-3′ (SEQ ID NO: 5), 5′-CTGC CCA CGG TAC CT-3′ (SEQ ID NO: 6), or 5′-AGACC CAG GCC CAC AT-3′ (SEQ ID NO: 7), or a complementary sequence thereof. In another embodiment, the oligonucleotide sequence is SEQ ID NO: 5, SEQ ID NO: 6, or SEQ ID NO: 7, or a complementary sequence thereof.

In another aspect, the present invention relates in part to methods for detecting the presence of ML IV mutant or wildtype sequences in a test sample. These methods preferably comprise a “real time PCR” assay providing dynamic fluorescence detection of amplified nucleic acid sequence of the MCOLNl gene, if present in the sample, using two or more pairs of oligonucleotide primers; hybridizing the amplified nucleic acids with one or more oligonucleotide probes specific for a Mucolipidosis IV mutant allele or a wildtype allele; and detecting a signal in real time from the hybridized nucleic acids, wherein the signal is related to the presence of the target sequences in the test sample.

In one embodiment, the method of determining the presence of a Mucolipidosis IV mutant sequence in a nucleic acid sample, comprises in any order contacting the nucleic acid with a) a first oligonucleotide primer that comprises a sequence complementary to a 15-30 bp segment of DNA between positions 5124-5524 of the MCOLN1 gene; b) a second oligonucleotide primer that comprises a sequence complementary to a 15-30 bp segment of DNA between positions 5541-5491 of the MCOLN1 gene; and c) an oligonucleotide probe that comprises a sequence complementary to a 13-30 bp segment of DNA that includes position 5534 between of the MCOLN1 gene, wherein the probe is labeled with a detectable label that comprises a donor fluorophore and a quencher moiety, wherein the quencher moiety is optionally an acceptor fluorophore. Once combined, amplification is conducted by temperature cycling and monitoring the accumulation of amplified nucleic in real time by detecting an increase in donor fluorophore fluorescence or a decrease in acceptor fluorophore fluorescence which indicates the presence of the Mucolipidosis IV mutant sequence in the nucleic acid.

In another embodiment, the method of determining the presence of a Mucolipidosis IV mutant sequence in a nucleic acid, comprises in any order the steps of contacting the nucleic acid sample with; a) a first oligonucleotide primer that comprises a sequence complementary to a 15-30 bp segment of DNA between positions 100-500 of the MCOLN1 gene, b) a second oligonucleotide primer that comprises a sequence complementary to a 15-30 bp segment of DNA between positions 6956-7356 of the MCOLN1 gene, and c) an oligonucleotide probe that comprises a sequence complementary to a 13-30 bp segment of a fragment that is amplified using the first and second oligonucleotide primers, wherein the probe is labeled with a detectable label that comprises a donor fluorophore and a quencher moiety, wherein the quencher moiety is optionally an acceptor fluorophore. Once combined, amplification is conducted by temperature cycling and monitoring the accumulation of amplified nucleic in real time by detecting an increase in donor fluorophore fluorescence or a decrease in acceptor fluorophore fluorescence which indicates the presence of the Mucolipidosis IV mutant sequence in the nucleic acid.

In yet another embodiment, the method of determining the presence of a Mucolipidosis IV mutant sequence in a nucleic acid, comprises in any order the steps of contacting the nucleic acid sample with; a) a first oligonucleotide primer that comprises a sequence complementary to a 15-30 bp segment of DNA between positions 100-500 of the MCOLN1 gene; b) a second oligonucleotide primer that comprises a sequence complementary to a 15-30 bp segment of DNA between positions 6956-7356 of the MCOLN1 gene, c) a first oligonucleotide probe that comprises a sequence complementary to a 13-30 bp segment of a fragment that is amplified using the first and second oligonucleotide primers, wherein the first probe is labeled with a first detectable label comprising a donor fluorophore and a quencher moiety wherein the quencher moiety is optionally an acceptor fluorophore; d) a third oligonucleotide primer that comprises a sequence complementary to a 15-30 bp segment of DNA between positions 100-500 of the MCOLN1 gene, e) a fourth oligonucleotide primer that comprises a sequence complementary to a 15-30 bp segment of DNA between positions 6956-7356 of the MCOLN1 gene, and f) a second oligonucleotide probe that comprises a sequence complementary to a 13-30 bp segment of a fragment that is amplified using the third and fourth primers, wherein the second probe is labeled with a second detectable label comprising a donor fluorophore and a quencher moiety, wherein the quencher moiety is optionally an acceptor fluorophore, and wherein the second detectable label is distinguishable from the first detectable label. Once combined, amplification is conducted by temperature cycling and monitoring the accumulation of amplified nucleic in real time by detecting an increase in donor fluorophore fluorescence or an increase or decrease in acceptor fluorophore fluorescence which indicates the presence of one or both of the Mucolipidosis IV mutant sequences in the nucleic acid.

In this latter method, the nucleic acid containing sample may also be contacted with a third oligonucleotide probe comprising a sequence consisting essentially of 5′-TCTG CCC ACA GTA CCT -3′ (SEQ ID NO: 5) that hybridizes to a wildtype sequence, wherein the third probe is labeled with a third detectable label comprising a donor fluorophore and a quencher moiety wherein the quencher moiety is optionally an acceptor fluorophore. Using this approach, the presence of a wildtype allele sequence can be determined to ascertain if an individual with a Mucolipidosis IV mutant sequence is homozygous or heterozygous for the mutation.

In the above methods, amplification by temperature cycling may be conducted using a DNA polymerase with 5′ exonuclease activity. In this approach, the specific binding of the probe to amplified nucleic acid results in degradation of the probe during DNA synthesis and separation of the donor fluorophore from the quencher moiety.

The presence of fluorescent signal from one or more of the labeled probes is detected simultaneously by a real-time instrument (e.g. ABI7900HT or Lightcycler) as presence of threshold cycle number or Ct value for the specific reporter probe.

In still yet another aspect, the present invention provides kits for one of the methods described herein. In various embodiments, the kits contain one or more of the following components in an amount sufficient to perform a method on at least one sample: one or more primers of the present invention, devices for performing the assay, which may include one or more probes that hybridize to mutant Mucolipidosis IV sequences and/or wildtype sequences, and optionally contain buffers, enzymes, and reagents for performing a method of detecting a genotype of Mucolipidosis IV in a nucleic acid of a sample.

### BRIEF DESCRIPTION OF THE FIGURES

FIG. 1 a diagram showing approximate locations of some primers and used to detect Mucolipidosis IV mutant sequences and wildtype sequences.

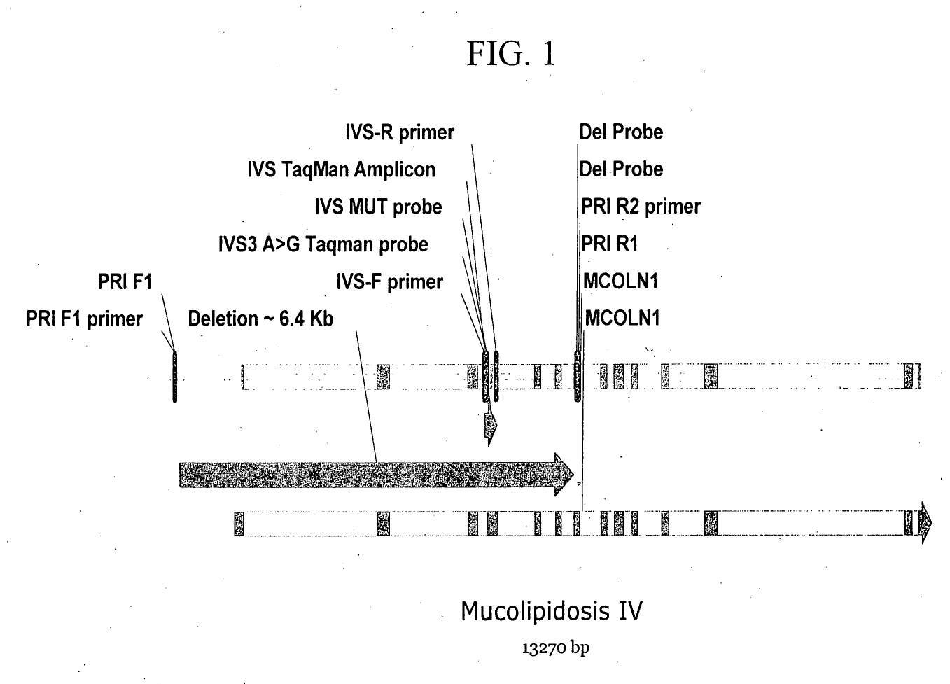

FIG. 2 is a diagram showing approximate locations of some primers and probes used to detect Mucolipidosis IV mutant sequences.

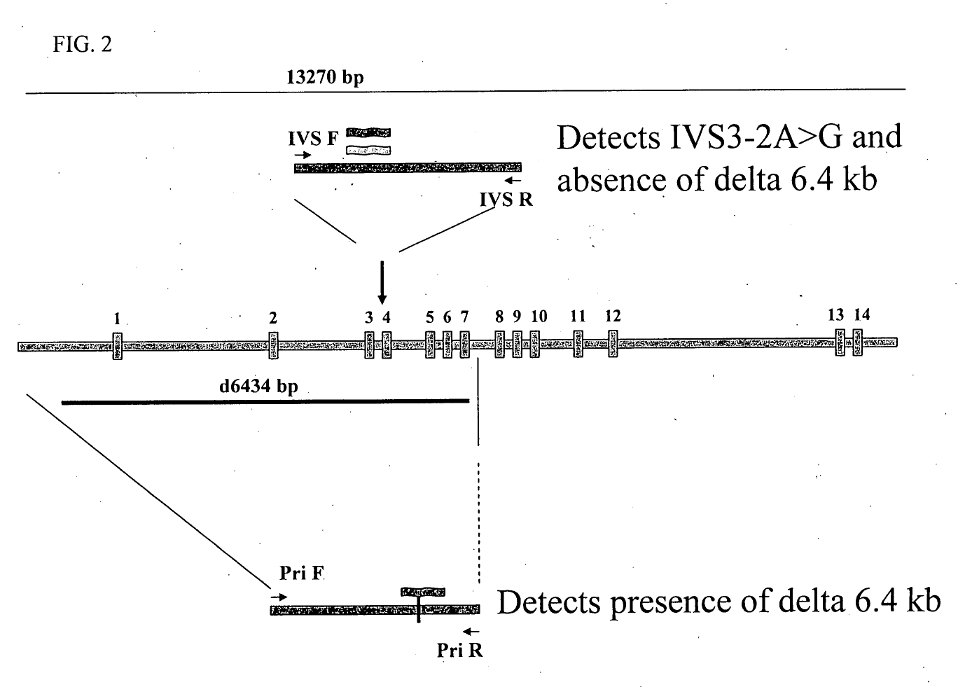

### DETAILED DESCRIPTION OF THE INVENTION

The present invention provides specific primers and probes that aid in the detection of ML IV mutant alleles or a wildtype allele. Primers are used to amplify one or more segments of the MCOLN1 gene. Probes are designed to detect an A>G transition (IVS 3-2 A>G), which causes a splice site mutation in the acceptor site of intron 3 is found in 72% of Ashkenazi ML IV alleles, and a 6.4 kb deletion that includes exons 1 to 7 of the MCOLN1 gene (GenBank accession no. AF287270). The primers and probes may be used in real time PCR. Simultaneous detection of two mutant alleles or two mutant alleles and a wildtype allele is made possible in real time PCR by labeling the probes with differentially detectable labels which comprise a donor fluorophore and a quencher moiety. The quencher moiety may optionally be an acceptor fluorophore. The separation of the donor fluorophore from the quencher resulting from a conformational change in the probe upon hybridization (e.g., molecular beacon probe) or from degradation of the probe by a 5′ nuclease activity of a DNA polymerase or reverse transcriptase in the PCR reaction (e.g., TaqMan® probe) reflects binding to amplified target and may be optically detected by an increase in emission of the donor fluorophore or by a decrease in emission of the acceptor fluorophore (it this feature is present in the quencher).

The term “mutant Mucolipidosis IV nucleic acid sequence,” “Mucolipidosis IV mutant sequences,” or “genotype for Mucolipidosis IV” as used herein means one or more nucleic acid sequences that are associated or correlated with ML IV. These mutant ML IV sequences may be correlated with a carrier state, or with a person afflicted with mucolipidosis type IV. The nucleic acid sequences are preferably DNA sequences, and are preferably genomic DNA sequences; however, RNA sequences such as mRNA or hnRNA may also contain nucleic acid sequences that are associated with mucolipidosis IV. Mucolipidosis IV mutations include A>G transition (IVS 3-2 A>G), which causes a splice site mutation in the acceptor site of intron 3 is found in 72% of Ashkenazi ML IV alleles, and a 6.4 kb deletion that includes exons 1 to 7 of the MCOLN1 gene (GenBank accession no. AF287270). Genotypes detectable using the invention methods include wt/wt, IVS/wt, Del/st, IVS/Del, IVS/IVS and Del/Del.

The term “MCOLN1 nucleic acid sequence” as used herein refers to a gene located on chromosome 19p13 that encodes the mucolipin-1 protein. Mucolipin-1 is a membrane protein with 6 transmembrane domains, a channel pore region, and a serine lipase motif. Mucolipin-1 shares homology with the calcium channels of the transient receptor potential (TRP) cation gene family.

The term “oligonucleotide” as used herein refers to a short polymer composed of deoxyribonucleotides, ribonucleotides or any combination thereof. These oligonucleotides are at least 9 nucleotides in length, preferably 15 to 70 nucleotides long, with 18 to 26 nucleotides being the most common. In certain embodiments, the oligonucleotides are chemically linked or otherwise associated with a detectable label.

The term “isolated” as used herein with reference to a nucleic acid (e.g., an RNA, DNA or a mixed polymer) refers to one which is substantially separated from other cellular components which naturally accompany such nucleic acid. The term embraces a nucleic acid sequence which has been removed from its naturally occurring environment, and includes recombinant or cloned DNA isolates, oligonucleotides, and chemically synthesized analogs or analogs biologically synthesized by heterologous systems.

The term “substantially pure” as used herein is a nucleic acid that represents more than 50% of the nucleic acid in a sample. The nucleic acid sample may exist in solution or as a dry preparation. Oligonucleotides, primers and probes of the invention are preferably substantially purified.

The term “hybridize” as used herein refers to process that two complementary nucleic acid strands anneal to each other under appropriately stringent conditions. Hybridizations are typically and preferably conducted with probe-length nucleic acid molecules, preferably 18-100 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

The term “substantially complementary” as used herein means that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target sequence, positioned 3′ or 5′ to a contiguous sequence of bases that hybridize under stringent hybridization conditions to a target sequence.

The term “flanking” as used herein means that a primer hybridizes to a target nucleic acid adjoining a region of interest sought to be amplified on the target. The skilled artisan will understand that preferred primers are pairs of primers that hybridize 3′ from a region of interest, one on each strand of a target double stranded DNA molecule, such that nucleotides may be add to the 3′ end of the primer by a suitable DNA polymerase.

The term “complement” as used herein means the complementary sequence to a nucleic acid according to standard Watson/Crick pairing rules. A complement sequence can also be a sequence of RNA complementary to the DNA sequence or its complement sequence, and can also be a cDNA.

The term “coding sequence” as used herein means a sequence of a nucleic acid or its complement, or a part thereof, that can be transcribed and/or translated to produce the mRNA for and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the encoding sequence can be deduced therefrom.

The term “non-coding sequence” as used herein means a sequence of a nucleic acid or its complement, or a part thereof, that is not transcribed into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, etc.

The term “amplification” or “amplify” as used herein means one or more methods known in the art for copying a target nucleic acid, thereby increasing the number of copies of a selected nucleic acid sequence. Amplification may be exponential or linear. A target nucleic acid may be either DNA or RNA. The sequences amplified in this manner form an “amplicon.” While the exemplary methods described hereinafter relate to amplification using the polymerase chain reaction (“PCR”), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al., Eds., Academic Press, San Diego, Calif. 1990, pp 13-20; Wharam et al., Nucleic Acids Res. 2001 June 1;29(11):E54-E54; Hafner et al., Biotechniques 2001 Apr;30(4):852-6, 858, 860 passim; Zhong et al., Biotechniques 2001 Apr;30(4):852-6, 858, 860 passim.

Exemplary primers for amplifying segments of the MCOLN1 gene are given in Table 1. Additional IVS forward primers include those that hybridize within a 400 bp stretch of DNA from position 5124 to 5524 (accession # AF287270). Additional IVS reverse primers include those that hybridize within a 400 bp stretch of DNA from position 5141 to 5941 (accession # AF287270).

Additional PRI forward primers include those that hybridize within a 400 bp stretch of DNA from position 100 to 500 (accession # AF287270). Additional PRI reverse primers include those that hybridize within a 400 bp stretch of DNA from position 6956 to 7356 (accession # AF287270

TABLE 1

MCOLN1 mutant and wildtype alleles and primers for
amplification

| | | Genbank accession # |
|---|---|---|
| Primer | Sequence | AF287270 |
| IVS-F | 5′-AGC GGG CCG GAC TCA-3′ | 5494-5509 |
| | (SEQ ID NO. 1) | |
| IVS-R | 5′-TAA CCA CCA TCG GAT CAA TGT C-3′ | 5671-5698 |
| | (SEQ ID NO. 2) | |
| PRI F1 | : 5′-CTT GCT CTG TTG CCC AGG CT -3′ | 441-460 |
| | (SEQ ID NO. 3) | |
| PRI R2 | 5′-CTC ACC GTG CTG GAA GAC ACT -3′ | 7017-7037 |
| | (SEQ ID NO. 4) | |

The term “probe” as used herein means a sequence of nucleic acid, preferably DNA, that hybridizes to a substantially complementary target sequence. The probe is attached to a detectable moiety. Probes generally comprise from 13 to 30 nucleotides, preferably from 14 to 25 nucleotides.

The term “detectable label” as used herein refers to a composition or moiety that is detectable by spectroscopic, photochemical, biochemical, immunochemical, electromagnetic, radiochemical, or chemical means such as fluorescence, chemifluoresence, or chemiluminescence, or any other appropriate means. Preferred detectable labels are fluorescent dye molecules, or fluorophores, such fluorescein, phycoerythrin, CY3, CY5, allophycocyanine, Texas Red, peridenin chlorophyll, cyanine, FAM, JOE, TAMRA, TET, VIC. These examples are not meant to be limiting. Methods and compositions for detectably labeling molecules, such as oligonucleotides, PNA-DNA hybrids, etc. are well known in the art. See, e.g., U.S. Pat. Nos. 6,316,230; 6,297,016; 6,316,610; 6,060,240; 6,150,107; and 6,028,290, each of which are hereby incorporated by reference in their entirety.

The term “linker” as used herein refers to one or more chemical bonds or a chemical group used to link one moiety to another, serving as a divalent bridge, where it provides a group between two other chemical moieties. A detectable label may be attached using a linker to the probe.

The term “identifying” as used herein with respect to an amplified sample is meant that the presence or absence of a particular nucleic acid amplification product is detected. Numerous methods for detecting the results of a nucleic acid amplification method are known to those of skill in the art.

The term “real time PCR” as used herein means that a signal emitted from the PCR assay is monitored during the reaction as an indicator of amplicon production during each PCR amplification cycle (i.e., in “real time”), as opposed to conventional PCR methods, in which an assay signal is detected at the endpoint of the PCR reaction. Real time PCR is generally based on the detection and quantitation of a fluorescent reporter. The signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. For a general description of “real time PCR” see Dehée et al. J. Virol. Meth. 102:37-51 (2002); and Aldea et al. J. Clin. Microbiol. 40:1060-1062 (2002) (referring to the “LightCycler,” where real-time, kinetic quantification allows measurements to be made during the log-linear phase of a PCR).

One general method for real time PCR uses fluorescent probes such as the TaqMang probes, molecular beacons and scorpions. Real-time reverse-transcriptase (RT) PCR quantitates the initial amount of the template with more specificity, sensitivity and reproducibility, than other forms of quantitative reverse transcriptase PCR, which detect the amount of final amplified product. Real-time RT-PCR does not detect the size of the amplicon. The probes employed in TaqMan® and molecular beacon technologies are based on the principle of fluorescence quenching and involve a donor fluorophore and a quenching moiety.

The term “fluorophore” as used herein refers to a molecule that absorbs light at a particular wavelength (excitation frequency) and subsequently emits light of a longer wavelength (emission frequency). The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e. collisional) quenching or by Förster or fluorescence resonance energy transfer (“FRET”). Quenching by FRET is generally used in TaqMan® probes while proximal quenching is used in molecular beacon and scorpion type probes.

In proximal quenching (a.k.a. “contact” or “collisional” quenching), the donor is in close proximity to the quencher moiety such that energy of the donor is transferred to the quencher, which dissipates the energy as heat as opposed to a fluorescence emission. In FRET quenching, the donor fluorophore transfers its energy to a quencher which releases the energy as fluorescence at a higher wavelength. Proximal quenching requires very close positioning of the donor and quencher moiety, while FRET quenching, also distance related, occurs over a greater distance (generally 1 -10 nm, the energy transfer depending on R⁻⁶, where R is the distance between the donor and the acceptor). Thus, when FRET quenching is involved, the quenching moiety is an acceptor fluorophore that has an excitation frequency spectrum that overlaps with the donor emission frequency spectrum. When quenching by FRET is employed, the assay may detect an increase in donor fluorophore fluorescence resulting from increased distance between the donor and the quencher (acceptor fluorophore) or a decrease in acceptor fluorophore emission resulting from increased distance between the donor and the quencher (acceptor fluorophore). TaqMan® probes (Heid et al., 1996) use the fluorogenic 5′ exonuclease activity of Taq polymerase to measure the amount of target sequences in cDNA samples. TaqMan® probes are oligonucleotides that contain a donor fluorophore usually at or near the 5′ base, and a quenching moiety typically at or near the 3′ base. The quencher moiety may be a dye such as TAMRA or may be a non-fluorescent molecule such as 4-(4-dimethylaminophenylazo)benzoic acid (DABCYL). See Tyagi et al., Nature Biotechnology 16:49-53 (1998). When irradiated, the excited fluorescent donor transfers energy to the nearby quenching moiety by FRET rather than fluorescing. Thus, the close proximity of the donor and quencher prevents emission of donor fluorescence while the probe is intact.

TaqMan® probes are designed to anneal to an internal region of a PCR product. When the polymerase (e.g.. reverse transcriptase) replicates a template on which a TaqMan® probe is bound, its 5′ exonuclease activity cleaves the probe. This ends the activity of quencher (no FRET) and the donor fluorophore starts to emit fluorescence which increases in each cycle proportional to the rate of probe cleavage. Accumulation of PCR product is detected by monitoring the increase in fluorescence of the reporter dye (note that primers are not labeled). If the quencher is an acceptor fluorophore, then accumulation of PCR product can be detected by monitoring the decrease in fluorescence of the acceptor fluorophore.

TaqMan® assay uses universal thermal cycling parameters and PCR reaction conditions. Because the cleavage occurs only if the probe hybridizes to the target, the fluorescence detected originates from specific amplification. The process of hybridization and cleavage does not interfere with the exponential accumulation of the product. One specific requirement for fluorogenic probes is that there be no G at the 5′ end. A ‘G’ adjacent to the reporter dye quenches reporter fluorescence even after cleavage.

Suitable donor fluorophores include 6-carboxyfluorescein (FAM), tetrachloro-6-carboxyfluorescein (TET), 2′-chloro-7′-phenyl-1,4-dichloro-6-carboxyfluorescein (VIC), and the like. Suitable quenchers include tetra-methylcarboxyrhodamine (TAMRA) 4-(4-dimethylaminophenylazo)benzoic acid (“DABCYL” or a DABCYL analog) and the like. Tetramethylrhodamine (TMR) or 5-carboxyrhodamine 6G (RHD) may be combined as donor fluorophores with DABCYL as quencher. Multiplex TaqMan assays can be performed using multiple detectable labels each comprising a different donor and quencher combination. Probes for detecting amplified sequence in real time may be stored frozen (−10° to −30° C.) as 100 [symbol] M stocks. TaqMan MGB probes are available from Applied BioSystems (4316032).

Exemplary probes for detecting amplified segments of the MCOLN1 gene as described herein are given in Table 2. The IVS wildtype or A>G transition probes may be extended or shortened on either end provided that the transition position (5534; Genbank accession no. AF287270) is not the end base of the probe. The probe for detecting the amplified fragment that results following deletion of 6.4 kb may hybridize anywhere within this amplified fragment. Using the outermost positions for the primers as described herein, the amplified fragment for the deletion mutant is about 800 bp assumes about and includes bases 100 to 500 followed by bases 6956 to 7356 (Genbank accession no. AF287270). A suitable probe in this case is one that consists essentially of from 13 to 30 nucleotides, preferably from 14 to 25 nucleotides of sequence anywhere in this approximately 800 pb of amplified DNA. Using the innermost positions for the primers as described herein and assuming a 40 base primer at each end, the amplified fragment for the deletion mutant is about 60 bp and includes bases 480 to 500 followed by bases 6956 to 6976 (Genbank accession no. AF287270). A suitable probe for this amplified fragment is one that consists essentially of from 13 to 30 nucleotides, preferably from 14 to 25 nucleotides of sequence anywhere in this approximately 60 pb of amplified DNA.

TABLE 2
Probes for Detection of wildtype and Mucolipidosis IV mutations

| ML IV | | |
|---|---|---|
| sequence target | Probe Name | Sequence |
| IVS Wildtype | IVS WT | 5′- TCTG CCC ACA GTA CCT -3′ |
| | | (SEQ ID NO: 5) |
| | | (Genbank nt positions 5525-5539; accession # |
| | | AF287270). The 5′ end is labeled with VIC. |
| IVS A > G | IVS MUT | 5′- CTGC CCA CGG TAC CT -3′ |
| transition (IVS | | (SEQ ID NO: 6) |
| 3-2 A > G) | | (Genbank nt positions 5526-5540; accession # |
| | | AF287270). The 5′ end is labeled with FAM. |
| 6.4 Kb deletion | DEL | 5′- AGACC CAG GCC CAC AT -3′ |
| of MCOLN1 | | (SEQ ID NO: 7) |
| including exons | | (Genbank nt positions 6982-6997; accession # |
| 1-7. | | AF287270). The 5′ end is labeled with TET. |

In a preferred embodiment, real time PCR is performed using TaqMan® probes in combination with a suitable amplification/analyzer such as the ABI Prism 7900HT Sequence Detection System. The ABI PRISM® 7900HT Sequence Detection System is a high-throughput real-time PCR system that detects and quantitates nucleic acid sequences. Briefly, TaqMan™ MGB probes specific for each allele are included in the PCR assay. These probes contain a reporter dye at the 5′ end and a quencher dye at the 3′ end. In addition, the minor-groove binding (MGB) component at the 3′ end of the probe stabilizes the specific hybridization of a TagMan™ probe to its DNA target and thus enhances the specificity of the assay. Each allele specific probe is conjugated with a different fluorescent reporter dye. During PCR, the fluorescently labeled probes bind specifically to their respective target sequences; the 5′ nuclease activity of Taq polymerase cleaves the reporter dye from the probe and a fluorescent signal is generated. The increase in fluorescence signal is detected only if the target sequence is complementary to the probe and is amplified during PCR. A mismatch between probe and target greatly reduces the efficiency of probe hybridization and cleavage. The ABI Prism 7900HT Sequence detection System measures the increase in fluorescence during PCR thermal cycling, providing “real time” detection of PCR product accumulation.

Real Time detection on the ABI Prism 7900HT Sequence Detector monitors fluorescence and calculates Rn during each PCR cycle. The threshold cycle, or Ct value, is the cycle at which fluorescence intersects the threshold value. The threshold value is determined by the sequence detection system software or manually.

As used herein, the term “sample” refers to any liquid or solid material believed to comprise nucleic acids containing the MCOLN1 gene or encoding the Mucolipin-1 protein. In preferred embodiments, a sample is obtained from a biological source, such as cells in culture or a tissue sample from an animal, most preferably, a human. Preferred sample tissues of the instant invention include, but are not limited to, plasma, serum, whole blood, blood cells, lymphatic fluid, cerebrospinal fluid, synovial fluid, urine, saliva, and skin or other organs (e.g. biopsy material). The term “patient sample” as used herein refers to a sample obtained from a human for which testing for MCOLN1 mutant alleles is desired.

Also included within the meaning of a “sample” is a biological specimen that has been processed to release or otherwise make available the nucleic acid for detection. For example, a biological sample may include a cDNA.

General sample volumes: Whole blood (5 ml per adult, 2 ml per child); amniotic Fluid (10-15 ml); cultured cells (two T-25 culture flasks with 80-100% confluent growth); extracted DNA (100 ng or more in TE or sterile water). and chorionic Villi (10-20 mg in a sterile container with 2-3 mL of sterile saline or tissue culture medium). Whole Blood may be collected preferably a lavender-top (EDTA) tube or yellow-top (ACD) tube. Green-top (Sodium Heparin) tubes may also be used but are not preferred.

Specimen Handling: Whole blood, amniotic fluid, cultured cells, and chorionic villi, are maintained preferably under ambient temperature (18-26° C.) while extracted DNA may be maintained for 24-48 hours (2-8° C.). Longer storage should be frozen. Samples are preferably analyzed without freezing.

Stability: Whole blood 8 days at ambient temperature (18-26° C.) or 8 days refrigerated (2-8° C.). Amniotic fluid, cultured cells, and chorionic villi: extract DNA within 24 hours of receipt. Extracted DNA is stable for 5 years refrigerated (2°-8° C.).

As used herein, “subject” means a human or any other animal which contains a MCOLN1 gene that can be amplified using the primers and methods described herein. A subject can be a patient, which refers to a human presenting to a medical provider for diagnosis or treatment of a disease. A human includes pre and post natal forms. Particularly preferred subjects are humans being tested for the existence of a Mucolipidosis carrier state or disease state.

The sample to be analyzed may consist of or comprise blood, sera, urine, feces, epidermal sample, skin sample, cheek swab, sperm, amniotic fluid, cultured cells, bone marrow sample and/or chorionic villi, and the like. A biological sample may be processed to release or otherwise make available a nucleic acid for detection as described herein. Such processing may include steps of nucleic acid manipulation, e.g., preparing a cDNA by reverse transcription of RNA from the biological sample. Thus, the nucleic acid to be amplified by the methods of the invention may be DNA or RNA.

The term “about” as used herein means in quantitative terms plus or minus 5%.

The term “substantially” as used herein means in quantitative terms 60% or more of the specified value or term.

The examples below illustrate a standard protocol for performing PCR and analyzing in real time. The TaqMan system of primer labeling is a preferred method of real time detection of PCR amplicons. The following examples serve to illustrate the present invention. These examples are in no way intended to limit the scope of the invention.

### EXAMPLE 1

### Detection of Mucolipidosis IV Mutant and Wildtype Alleles from Whole Blood

A. Extraction of DNA

Suitable samples may include fresh tissue, e.g., obtained from clinical swabs from a region where cells are collected by soft abrasion (e.g., buccal, cervical, vaginal, etc. surfaces) or biopsy specimens; cells obtained by amniocentesis or chorionic villus sampling; cultured cells, or blood cells; or may include fixed or frozen tissues. The following example describes preparation of nucleic acids from blood.

5 mL of whole blood was collected was 0.5 ml of TE (10 mM Tris HCl, 1 mM EDTA, pH 7.5). The sample was processed to obtain genomic DNA.

B. Amplification from DNA

Individual amplifications were prepared in a volume of 50 [symbol] 1, which was added to 96 well microtiter plates. Each amplification volume contained 4 [symbol] 1 of the DNA sample (generally 80-160 ng of DNA) and 46 [symbol] 1 of working PCR master mix. Working PCR master mix comprised 25 μl of TaqMan 2× Universal Master Mix (Applied Biosystems, #4304437) and 21 [symbol] 1 of Mucolipidosis IV primer/probe mix. Mucolipidosis IV primer/probe mix was prepared using 100 [symbol] M stock primers: IVS-F (SEQ ID NO: 1); IVS-R (SEQ ID NO:2); PRI-F1 (SEQ ID NO: 3); and PRI-R2 (SEQ ID NO: 4); 100 [symbol] M TaqMan probes: (Cat #s: 4316034, 4316033, 4316032, from Applied BioSystems. The fluorescent label is specified when entering the sequences); IVS-WT probe (SEQ ID NO: 5); labeled with VIC; IVS-MUT probe (SEQ ID NO: 6) labeled with FAM; and DEL probe (SEQ ID NO: 7) labeled with TET; 25 mM MgCl₂ (Qiagen #203205); 5× Q solution (Qiagen #203205); 100× BSA (EB B90015); and molecular biology grade water (Bio Whittaker #16-001Y). Mucolipidosis IV primer/probe mix was prepared from these reagents as indicated in Table 3.

TABLE 3
Mucolipidosis IV primer/probe mix

| | Final | Per rxn. | Cocktail |
|---|---|---|---|
| Reagent | Conc. | (μL) | (μL) × 100 |
| Nuclease-free dH₂O | NA | 9.4 | 940 |
| IVS-F Primer (100 [symbol] M) | 0.9 μM | 0.45 | 45 |
| IVS-R Primer (100 [symbol] M) | 0.9 μM | 0.45 | 45 |
| PRI F1 Primer (100 [symbol] M) | 0.9 μM | 0.45 | 45 |
| PRI R2 Primer (100 [symbol] M) | 0.9 μM | 0.45 | 45 |
| 25 mM Mg | 0.5 μM | 1 | 100 |
| 5 × Q Solution | 4× | 2.5 | 250 |
| 100 × BSA | 2× | 1 | 100 |
| IVS WT probe (10 [symbol] M) | 0.2 μM | 1 | 100 |
| IVS MUT probe (10 [symbol] M) | 0.36 μM | 1.8 | 180 |
| DelProbel (10 [symbol] M) | 0.5 μM | 2.5 | 250 |
| Total | | 21 | 2100 |

NA = non-applicable.

The following controls were used:

Positive and negative Control DNA: Six sets of positive DNA control DNAs were used which include the following genotypes wt/wt, IVS/wt, Del/st, IVS/Del, IVS/IVS and Del/Del. Positive DNA was obtained from previously tested DNA samples or was obtained from a cell repository (Coriell).

Negative controls included the following;
a) NS Control: a reagent blank (NS control) comprises all reagents and processing used to prepare sample DNA but without any starting DNA; and
b) ND Control: A minus DNA control (ND control) is used which consists of a PCR kit and polymerase mix used for the assay run.

Positional Control: a QC blank is placed randomly within each plate to ensure results reflect the correct positioning of the Extraction/PCR plate for detection.

Following addition of DNA and working PCR master mix, wells were tightly sealed with an optically transparent adhesive cover. The plate was centrifuged at ˜1600 rpm for 15 seconds to ensure that the added volumes were combined.

The plate was subject to PCR with real time analysis using an ABI 7900 HT instrument according to the manufacturing instructions. PCR was conducted using the following temperature profile:

TABLE 4
PCR temperature profile

| Step | Temp | Time |
|---|---|---|
| 1 | 50° C. | 2 min |
| 2 | 95° C. | 10 min |
| 3 | 60° C. | 1 min |
| 4 | Go to Step 2 | 44 × times |
| 5 | 4° C. | Hold |

C. Detection of Muycolipidosis IV Mutant Alleles

Real time detection on the ABI PRISM 7900HT sequence detector monitors fluorescence during amplification and calculates Rn during each cycle. Rn is the difference between report fluorescence and background in the sample. The threshold cycle or Ct value is the cycle at which flourescence intersects the threshold value. The threshold value is determined by the sequence detection software or manually.

The results were analyzed using software available with the AVI PRISM 7900HT Sequence Detection System, using version 2.0 software or higher. Data are collected are then analyzed automatically by two different methods. The cycle threshold values and the fluorescence signals generated and reported by the multicomponent results and the processed genotype results are downloaded into a laboratory information management system with the following output.

Using Ct Value: Using the Ct values, a logical statement can be fed into an Excel macro. A suitable equation is:

=IF(AND(X₁<35, Y₁>39, Z₁>39),“wt/wt”,IF(AND(X₁<35, Y₁<35,

Z₁>39),“IVS/wt”,IF(AND(X₁>40, Y₁<35, Z₁>39),“IVS/IVS”,IF(AND(X₁<35, Y₁>39,

Z₁<35),“del/wt”,IF(AND(X₁>39, Y₁>39, Z₁<35),“del/del”,IF(AND(X₁>39, Y₁<35,

Z₁<35),“IVS/del”,“Repeat!”))))))

where X₁, Y₁, Z₁, refer to the Ct values of the VIC, FAM and TET probes, respectively, from patient sample in well # 1. The numerical values for each signal (e.g. X1<35) indicate the Ct value for the particular probe is less than 35 cycles. Results were compared to wt/wt and NS controls.

Using The Ct Values (results.txt output) and The multicomponent results fluorescence values (multi.txt output):

# wt/wt

1. Only VIC is not undetermined.

2. the fam/vic is ratio less than or equal to 1.0

3. the tet/vic is ratio less than or equal to 1.0

# del6.4 kb homo

1. Both FAM and VIC are undetermined.

2. the fam/vic ratio is less than or equal to 1.0

3. the tet/vic ratio is between 1.9 and 5.0 inclusively.

# del6.4 kb/wt

1. Only FAM is undetermined.

2. the fam/vic ratio is less than or equal to 1.0

3. the tet/vic ratio is between 1.0 and 5.0 inclusively.

# del6.4 kb/IVS3-1 A>G

1. Only VIC is undetermined.

2. the fam/vic ratio is greater than or equal to 1.0

3. the tet/vic ratio is between 1.0 and 5.0 inclusively.

# IVS3-1 A>G homo

1. Both TET and VIC are undetermined

2. the fam/vic ratio is greater than or equal to 1.9

3. the tet/vic ratio is less than or equal 1.0.

# IVS3-1 A>G/wt

1. Only TET is undetermined

2. the fam/vic ratio is less than or equal to 1.5

3. the tet/vic ratio is less than or equal 1.5.

Criteria 1 is based on *result2.txt. (Ct values)

Criteria 2 and 2 are based on *multi.txt (multicomponent)

a) Neg:

Result: The patient is negative for the IVS3-2A>G and 511 del 6.4 kb mutations in the Mucolipin (MCOLN1) gene.

b) IVS3Het:

Result: The patient is heterozygous for the IVS3-2 A>G ML IV mutation and negative for the 511 del 6.4 kb ML IV mutation.

c) IVS3homo:

Result: The patient is homozygous for the IVS3-2 A>G ML IV mutation.

d) IVS3/DelCHet:

Result: The patient is a compound heterozygote for the IVS3-2 A>G and the 511 del 6.4 kb ML IV mutations.

e) delHet:

Result: The patient is heterozygous for the 511 del 6.4 kb ML IV mutation and negative for the IVS3-2 A>G ML IV mutation.

f) delhomo:

Result: This patient is homozygous for the 511 del 6.4 kb ML IV mutation.

Assay tolerance limits: Positive control DNA should exhibit the correct genotype. If more than one pair of positive controls fail to yield the correct genotype, the entire assay should be repeated. Negative controls should display no significant amplification. If the NS control shows evidence of significant amplification, all the patient samples associated with that NS control are potentially contaminated. If the ND control yields significant amplification, the PCR amplification reagents are potentially contaminated. Depending on the results, the specimens may need to be re-extracted and re-assayed (NS positive) or the entire assay must be repeated (ND positive). The QC Blank control should display no significant signal. If this blank shows evidence of a PCR product, the entire assay, or portions thereof, may need to be repeated.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. The term “consisting essentially of when used in connection with nucleotide sequence shall mean the specified sequence and other sequences which have relatively minor differences that do not specifically (i.e., by more than 50%) affect the function of the sequence for its intended purpose. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

1. An oligonucleotide comprising a sequence complementary to the coding or non-coding strand of the MCOLN1 gene wherein said sequence includes a sequence consisting essentially of: 5′-AGC GGG CCG GAC TCA-3′ (SEQ ID NO. 1), 5′-TAA CCA CCA TCG GAT CAA TGT C-3′ (SEQ ID NO. 2), 5′-CTT GCT CTG TTG CCC AGG CT-3′(SEQ ID NO. 3), or 5′-CTC ACC GTG CTG GAA GAC ACT-3′ (SEQ ID NO. .4), or a complementary sequence thereof.

2. An oligonucleotide of claim 1 wherein said sequence is SEQ ID NO. 1, SEQ ID NO. 2, SEQ ID NO. 3; or SEQ ID NO. 4, or a complementary sequence thereof.

3. An oligonucleotide comprising a sequence complementary to the coding or non-coding strand of the MCOLN1 gene wherein said sequence includes a sequence consisting essentially of: 5′-TCTG CCC ACA GTA CCT -3′ (SEQ ID NO: 5), 5′-CTGC CCA CGG TAC CT-3′ (SEQ ID NO: 6), or 5′-AGACC CAG GCC CAC AT-3′ (SEQ ID NO: 7), or a complementary sequence thereof.

4. The oligonucleotide of claim 4 wherein said sequence is SEQ ID NO: 5, SEQ ID NO: 6, or SEQ ID NO: 7, or a complementary sequence thereof.

5. The oligonucleotide of claim 1 wherein the oligonucleotide is conjugated to a detectable label.

6. The oligonucleotide of claim 3 wherein the oligonucleotide is conjugated to a detectable label.

7. The oligonucleotide of claim 6 wherein the detectable label comprises a donor fluorophore and quencher moiety.

8. The oligonucleotide of claim 7 wherein the donor fluorophore is 2′-chloro-7′-phenyl-1,4-dichloro-6-carboxyfluorescein (VIC), 6-carboxyfluorescein (FAM) or tetrachloro-6-carboxyfluorescein (TET).

9. The oligonucleotide of claim 8 wherein the quencher moiety is tetra-methylcarboxyrhodamine (TAMRA) or 4-(4′-dimethylaminophenylazo)benzoic acid (DABCYL).

10. The oligonucleotide of claim 8 wherein the sequence is 5′-TCTG CCC ACA GTA CCT -3′ (SEQ ID NO: 5) and the molecular beacon pair is VIC and a quencher moiety.

9. The oligonucleotide of claim 8 wherein the sequence is 5′-CTGC CCA CGG TAC CT-3′ (SEQ ID NO: 6) and the donor fluorophore is FAM.

10. The oligonucleotide of claim 8 wherein the sequence is 5′-AGACC CAG GCC CAC AT-3′ (SEQ ID NO: 7) and the donor fluorophore is TET.

11. A method of determining the presence of a Mucolipidosis IV mutant sequence in a nucleic acid, comprising,
a) contacting the nucleic acid with:
i) a first oligonucleotide primer that comprises a sequence complementary to a 15-30 bp segment of DNA between positions 5124-5524 of the MCOLN1 gene;
ii) a second oligonucleotide primer that comprises a sequence complementary to a 15-30 bp segment of DNA between positions 5541-5491 of the MCOLN1 gene,
iii) an oligonucleotide probe that comprises a sequence complementary to a 13-30 bp segment of DNA that includes position 5534 between of the MCOLN1 gene, wherein said probe is labeled with a detectable label which comprises a donor fluorophore and a quencher moiety, wherein said quencher moiety is optionally an acceptor fluorophore; and
b) conducting amplification by temperature cycling and monitoring the accumulation of amplified nucleic in real time by detecting an increase in donor fluorophore fluorescence or a decrease in acceptor fluorophore fluorescence which indicates the presence of the Mucolipidosis IV mutant sequence in the nucleic acid.

12. The method of claim 11 wherein the first oligonucleotide primer comprises a sequence that consists essentially of 5′-AGC GGG CCG GAC TCA-3′ (SEQ ID NO. 1).

13. The method of claim 11 wherein the second oligonucleotide primer comprises a sequence that consists essentially of 5′-TAA CCA CCA TCG GAT CAA TGT C-3′ (SEQ ID NO. 2).

14. The method of claim 11 wherein the probe comprises a sequence that consists essentially of 5′-CTGC CCA CGG TAC CT-3′ (SEQ ID NO: 6).

15. The method of claim 11 wherein the donor fluorophore is 2′-chloro-7′-phenyl-1,4-dichloro-6-carboxyfluorescein (VIC), 6-carboxyfluorescein (FAM) or tetrachloro-6-carboxyfluorescein (TET).

16. The method of claim 11 wherein amplification by temperature cycling is with a DNA polymerase with 5′ exonuclease activity and wherein binding of the probe to amplified nucleic acid results in degradation of the probe during DNA synthesis.

17. A method of determining the presence of a Mucolipidosis IV mutant sequence in a nucleic acid, comprising,
a) contacting the nucleic acid with;
i) a first oligonucleotide primer that comprises a sequence complementary to a 15-30 bp segment of DNA between positions 100-500 of the MCOLN1 gene,
ii) a second oligonucleotide primer that comprises a sequence complementary to a 15-30 bp segment of DNA between positions 6956-7356 of the MCOLN1 gene,
iii) an oligonucleotide probe that comprises a sequence complementary to a 13-30 bp segment of a fragment that is amplified using the first and second primer, wherein said probe is labeled with a detectable label which comprises a donor fluorophore and a quencher moiety, wherein said quencher moiety is optionally an acceptor fluorophore;
b) conducting amplification by temperature cycling and monitoring the accumulation of amplified nucleic in real time by detecting an increase in donor fluorophore fluorescence or a decrease in acceptor fluorophore fluorescence which indicates the presence of the Mucolipidosis IV mutant sequence in the nucleic acid.

18. The method of claim 17 wherein the first oligonucleotide primer comprises a sequence that consists essentially of 5′-CTT GCT CTG TTG CCC AGG CT-3′(SEQ ID NO. 3).

19. The method of claim 17 wherein the second oligonucleotide primer comprises a sequence that consists essentially of and 5′-CTC ACC GTG CTG GAA GAC ACT-3′ (SEQ ID NO. 4).

20. The method of claim 17 wherein the probe comprises a sequence that consists essentially of and the probe in ii) is 5′-AGACC CAG GCC CAC AT-3′ (SEQ ID NO: 7).

21. The method of claim 17 wherein the donor fluorophore is 2′-chloro-7′-phenyl-1,4-dichloro-6-carboxyfluorescein (VIC), 6-carboxyfluorescein (FAM) or tetrachloro-6-carboxyfluorescein (TET).

22. The method of claim 17 wherein amplification by temperature cycling is with a DNA polymerase with 5′ exonuclease activity and wherein binding of the probe to amplified nucleic acid results in degradation of the probe during DNA synthesis.

23. A method of detecting the presence of one or two Mucolipidosis IV mutant sequences in a nucleic acid, comprising,
a) contacting the nucleic acid with;
i) a first oligonucleotide primer that comprises a sequence complementary to a 15-30 bp segment of DNA between positions 100-500 of the MCOLN1 gene,
ii) a second oligonucleotide primer that comprises a sequence complementary to a 15-30 bp segment of DNA between positions 6956-7356 of the MCOLN1 gene,
iii) a first oligonucleotide probe that comprises a sequence complementary to a 13-30 bp segment of a fragment that is amplified using the first and second oligonucleotide primers, wherein said probe is labeled with a first detectable label which comprises a donor fluorophore and a quencher moiety, wherein said quencher moiety is optionally an acceptor fluorophore;
iv) a third oligonucleotide primer that comprises a sequence complementary to a 15-30 bp segment of DNA between positions 100-500 of the MCOLN1 gene,
v) a fourth oligonucleotide primer that comprises a sequence complementary to a 15-30 bp segment of DNA between positions 6956-7356 of the MCOLN1 gene, and
vi) a second oligonucleotide probe that comprises a sequence complementary to a 13-30 bp segment of a fragment that is amplified using the third and fourth primers, wherein said probe wherein said probe is labeled with a second detectable label which comprises a donor fluorophore and a quencher moiety, wherein said quencher moiety is optionally an acceptor fluorophore, and wherein said second detectable label is distinguishable from said first detectable label;
b) conducting amplification by temperature cycling and monitoring the accumulation of amplified nucleic in real time by detecting an increase in donor fluorophore fluorescence or an increase or decrease in acceptor fluorophore fluorescence, which indicates the presence of one or both of the Mucolipidosis IV mutant sequences in the nucleic acid.

24. The method of claim 23 wherein the first oligonucleotide primer comprises a sequence that consists essentially of 5′-AGC GGG CCG GAC TCA-3′ (SEQ ID NO. 1).

25. The method of claim 23 wherein the second oligonucleotide primer comprises a sequence that consists essentially of 5′-TAA CCA CCA TCG GAT CAA TGT C-3′ (SEQ ID NO. 2).

26. The method of claim 23 wherein the first probe comprises a sequence that consists essentially of 5′-CTGC CCA CGG TAC CT -3′ (SEQ ID NO: 6).

27. The method of claim 23 wherein the third oligonucleotide primer comprises a sequence that consists essentially of 5′-CTT GCT CTG TTG CCC AGG CT-3′(SEQ ID NO. 3).

28. The method of claim 23 wherein the fourth oligonucleotide primer comprises a sequence that consists essentially of 5′-CTC ACC GTG CTG GAA GAC ACT -3′ (SEQ ID NO. 4).

28. The method of claim 23 wherein the second probe comprises a sequence that consists essentially of 5′-AGACC CAG GCC CAC AT-3′ (SEQ ID NO: 7).

30. The method of claim 23 wherein the first or second donor fluorophore is 2′-chloro-7′-phenyl-1,4-dichloro-6-carboxyfluorescein (VIC), 6-carboxyfluorescein (FAM) or tetrachloro-6-carboxyfluorescein (TET).

31. The method of claim 23 wherein amplification by temperature cycling is with a DNA polymerase with 5′ exonuclease activity and wherein binding of the probe to amplified nucleic acid results in degradation of the probe during DNA synthesis.

32. The method of claim 23 wherein said nucleic acid containing sample is also contacted with a third oligonucleotide probe comprising a sequence consisting essentially of 5′-TCTG CCC ACA GTA CCT-3′ (SEQ ID NO: 5) that hybridizes to a wildtype sequence, wherein said third probe is labeled with a detectable label comprising a donor fluorophore and a quencher moiety wherein said quencher moiety is optionally an acceptor fluorophore, and wherein said third detectable label is distinguishable from said first and second detectable labels.

33. A kit for amplifying sequences of Mucolipidosis IV sequences comprising one or more oligonucleotides of claim 1.

31. The kit of claim 32 further comprising one or more oligonucleotides of claim 3.
